(19) United States
(12) Patent Application Publication
(10) Pub. No.: US 2009/0163800 A1
(43) Pub. Date: Jun. 25, 2009

(54) TOOLS AND METHODS FOR VISUALIZATION AND MOTION COMPENSATION DURING ELECTROPHYSIOLOGY PROCEDURES

(75) Inventors: Chenyang Xu, Allentown, NJ (US); Rui Liao, Plainsboro, NJ (US); Liron Yatziv, Fremont, CA (US); Norbert Strobel, Heroldsbach (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Corporate Research, Inc., Princeton, NJ (US)

(21) Appl. No.: 12/335,738

(22) Filed: Dec. 16, 2008

Related U.S. Application Data
(60) Provisional application No. 61/015,427, filed on Dec. 20, 2007.
(60) Provisional application No. 61/086,249, filed on Aug. 5, 2008.

(51) Int. Cl. A61B 6/00; A61B 5/05

(52) U.S. Cl. 600/424; 600/427

(57) ABSTRACT

A method for real-time cardiac visualization includes acquiring fluoroscope imagery from two planes. The location of at least one electrophysiology (EP) device is marked within the fluoroscope imagery from each of the two planes. The location information for the at least one EP device is combined within each of the acquired fluoroscope images from the two planes to determine a 3D location for the at least one EP device. The fluoroscope imagery from at least one of the two planes is displayed with a visual aid superimposed thereon. The visual aid is based on the 3D location of the EP device.

### CROSS-REFERENCE TO RELATED APPLICATION

The present application is based on provisional application Ser. No. 61/015,427, filed Dec. 20, 2007 and provisional application Ser. No. 61/086,249, filed Aug. 5, 2008, the entire contents of which are herein incorporated by reference.

### BACKGROUND OF THE INVENTION

1. Technical Field

The present disclosure relates to electrophysiology procedures and, more specifically, to tools and methods for visualization and motion compensation during electrophysiology procedures.

2. Discussion of Related Art

Electrophysiology (EP) is the study of the electrical properties of biological tissue such as the human heart. In EP, electrodes may be placed in various locations around the biological tissue being studied to monitor the exchange of electrical signals. Electrocardiography is the study of the electrical properties of the human heart. Because in the clinical environment, the human heart is most often the subject of EP studies, electrocardiography is often simply referred to as EP.

The most common electrocardiographic test is the electrocardiogram (ECG). The ECG is a recording of the electrical activity of the heart as observed by an electrocardiograph. This test may be non-invasive as electrodes may be selectively placed on the skin of the subject. The recorded electrical signals may provide a medical practitioner with insight into the rhythm of the heart and potential weaknesses of different parts of the heart.

Where greater particularity is required, more invasive EP procedures may be performed by placing electrodes inside the human body and indeed inside of the heart, where needed. In order to accurately place the electrodes, it may be necessary to visualize the heart using a medical imaging device. As the heart is constantly in motion, and the location of tools in and around the heart must be known, fluoroscopy is often used to visualize the tools, the heart, and the surrounding region.

Fluoroscopy is an imaging technique that relies on x-rays to provide a continuing series of images that provides a real-time moving image of the area being visualized. In fluoroscopy, the resulting image is a two-dimensional representation of the area being visualized, wherein anatomical features may be visible without an accurate sense of depth.

Radio-frequency (RF) catheter ablation may also be performed in combination with the invasive EP procedures discussed above. In RF catheter ablation, an RF catheter may be used to destroy abnormal electrical pathways in heart tissue. This procedure may be used to treat atrial fibrillation and other forms of cardiac arrhythmia.

RF catheter ablation may be used in concert with invasive EP procedures so that abnormal electrical pathways can be precisely located prior to ablation, and the effectiveness of the ablation can be judged prior to ending the procedure. For these reasons, fluoroscopy may be used to provide a real-time visualization for both EP procedures and RF catheter ablation.

Because of the lack of depth associated with the two-dimensional-fluoroscope imagery, medical practitioners performing invasive EP procedures and RF catheter ablation guided by fluoroscope imagery may have a difficult time locating electrodes, RF catheters and other tools to the vicinity of various anatomical structures. For example, it may be especially difficult for medical practitioners to interact with the four pulmonary veins that carry oxygenated blood from the lungs to the left atrium of the heart.

Recently techniques have been developed for fusing the fluoroscope imagery with high-resolution three-dimensional atrial CT and/or MR volumes to augment the moving real-time fluoroscope imagery with the detailed three-dimensional structural data of a reference CT and/or MR volume that may be acquired prior to performing the fluoroscopy. However, because the heart is constantly in motion as a result of the cardiac cycle and breathing, it can be difficult to maintain proper registration of the fluoroscope imagery and the volume data throughout the cardiac cycle and throughout the various respiratory phases. While cardiac motion may be compensated for using ECG gating, breathing motion is less periodic than cardiac motion and thus it can be particularly difficult to compensate for breathing.

Additionally, when fused fluoroscopy is used, it is necessary that the structure of the heart within the fluoroscope imagery be accurately matched to the corresponding structure of the heart within the volume data. This initial registration should be performed in three-dimensions, and as described above, this can be a difficult task given the fact that the two-dimensional fluoroscope imagery lacks a perspective of depth.

Accordingly, when performing initial registration of fluoroscope imagery to volume data from a CT and/or MR scan, and when performing invasive EP procedures and/or catheter ablation from un-fused fluoroscope imagery, it can be difficult to find key structural elements such as the pulmonary veins without depth information. Moreover, when performing invasive EP procedures and/or catheter ablation from fused imagery, it can be difficult to maintain proper registration during breathing.

### SUMMARY

A method for real-time cardiac visualization includes acquiring fluoroscope imagery from two planes. The location of at least one electrophysiology (EP) device is marked within the fluoroscope imagery from each of the two planes. The location information for the at least one EP device is combined within each of the acquired fluoroscope images from the two planes to determine a 3D location for the at least one EP device. The fluoroscope imagery from at least one of the two planes is displayed with a visual aid superimposed thereon. The visual aid is based on the 3D location of the EP device.

Acquiring the fluoroscope imagery from two planes may include acquiring fluoroscope imagery from a first plane using an x-ray detector, repositioning the x-ray detector to a second plane, and acquiring fluoroscope imagery from the second plane using the repositioned x-ray detector. Alternatively, acquiring the fluoroscope imagery from two planes may include acquiring fluoroscope imagery from a first plane using a first x-ray detector, and acquiring fluoroscope imagery from a second plane using a second x-ray detector. The first x-ray detector and the second x-ray detector may be part of a single biplane fluoroscope.

The location of the at least one EP device may be marked manually by a user who is presented with an on-screen representation of each fluoroscope image and selects the location of the EP device on each fluoroscope image. Alternatively, the location of the at least one EP device is marked automatically on each fluoroscope image using computer vision techniques.

The EP device(s) may be made up of a lasso catheter and/or a CS catheter.

Displaying the fluoroscope imagery from at least one of the two planes with a visual aid superimposed thereon may include displaying a shape marker indicating the 3D location of a pulmonary vein edge. The shape marker may be an ellipse.

Alternatively, or additionally, displaying the fluoroscope imagery from at least one of the two planes with a visual aid superimposed thereon may include displaying a shape marker indicating the 3D location of the at least one EP device.

Alternatively, or additionally, displaying the fluoroscope imagery from at least one of the two planes with a visual aid superimposed thereon may include displaying a suggested ablation path.

Alternatively, or additionally, displaying the fluoroscope imagery from at least one of the two planes with a visual aid superimposed thereon may include displaying a rendered 3D segmentation of a left atrium.

A method for compensating for breathing motion in a real-time cardiac visualization includes acquiring fluoroscope imagery from two planes. At least one electrophysiology (EP) device is tracked within the acquired fluoroscope imagery from each of the two planes. A 2D trajectory is constructed for the at least one EP device within the acquired fluoroscope imagery from each of the two planes based on the tracking. A 3D trajectory is constructed for the at least one EP device by combining the 2D trajectories of the at least one EP device for each of the two planes. A breathing motion is determined based on the constructed 3D trajectory. The determined breathing motion is compensated for within the acquired fluoroscope imagery.

The acquired fluoroscope imagery may be registered to 3D volume data acquired from a CT or MR and the fluoroscope imagery may be fused to the registered 3D volume data such that the fused image data provides a real-time moving image with structural detail. The fused image data may be compensated for by the determined breathing motion.

Fusing the fluoroscope imagery to the registered 3D volume data may include matching the fluoroscope imagery to the cardiac phase of the 3D volume data and performing ECG.

Performing initial registration of the fluoroscope imagery to the 3D volume data may include marking the location of at least one EP device within the fluoroscope imagery from each of the two planes, combining the location information for the at least one EP device within each of the acquired fluoroscope images from the two planes to determine a 3D location for the at least one EP device, identifying a 3D location of an anatomical structure within the fluoroscope imagery based on the determined 3D location for the at least one EP device, and registering the fluoroscope imagery to the 3D volume using the identified 3D location of the anatomical structure.

Acquiring the fluoroscope imagery from two planes may include acquiring fluoroscope imagery from a first plane using an x-ray detector, repositioning the x-ray detector to a second plane, and acquiring fluoroscope imagery from the second plane using the repositioned x-ray detector.

The fluoroscope imagery from two planes may include acquiring fluoroscope imagery from a first plane using a first x-ray detector, and acquiring fluoroscope imagery from a second plane using a second x-ray detector, wherein the first x-ray detector and the second x-ray detector are part of a single biplane fluoroscope.

The at least one EP device may include a lasso catheter and/or a CS catheter.

A computer system includes a processor and a program storage device readable by the computer system, embodying a program of instructions executable by the processor to perform method steps for real-time cardiac visualization. The method includes acquiring fluoroscope imagery from two planes, marking the location of at least one lasso catheter within the fluoroscope imagery from each of the two planes, combining the location information for the at least one lasso catheter within each of the acquired fluoroscope images from the two planes to determine a 3D location for the at least one lasso catheter, determining the 3D location of one or more pulmonary vein edges based on the determined 3D location of the at least one lasso catheter, and displaying the fluoroscope imagery from at least one of the two planes with an indication of the 3D location of the one or more pulmonary vein edges superimposed thereon.

Acquiring the fluoroscope imagery from two planes may include acquiring fluoroscope imagery from a first plane using an x-ray detector, repositioning the x-ray detector to a second plane, and acquiring fluoroscope imagery from the second plane using the repositioned x-ray detector.

Acquiring the fluoroscope imagery from two planes may include acquiring fluoroscope imagery from a first plane using a first x-ray detector, and acquiring fluoroscope imagery from a second plane using a second x-ray detector, wherein the first x-ray detector and the second x-ray detector are part of a single biplane fluoroscope.

A method for real-time cardiac visualization includes acquiring fluoroscope imagery from a single plane with a stationary x-ray detector, marking the location of at least one electrophysiology (EP) device within the fluoroscope imagery, determining a location for the at least one EP device based on the fluoroscope imagery and a location of the stationary x-ray detector, and displaying the fluoroscope imagery with a graphical visual aid superimposed thereon, the visual aid being based on the location of the EP device.

The location of the at least one EP device may be marked manually by a user who is presented with an on-screen representation of the fluoroscope image and selects the location of the EP device on the fluoroscope image or may be marked semi-automatically with the use of an interactive tool. The location of the at least one EP device may be marked automatically on the fluoroscope image using computer vision techniques. The at least one EP device may include a lasso catheter or a CS catheter. Displaying the fluoroscope imagery with a visual aid superimposed thereon may include displaying a shape marker indicating the location of a pulmonary vein edge. The he shape marker may be an ellipse.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a flow chart illustrating a method for visualization using a bi-plane fluoroscope according to an exemplary embodiment of the present invention;

FIG. 2A is a diagram of a heart with a lasso catheter placed on a pulmonary vein edge according to an exemplary embodiment of the present invention;

FIG. 2B is a sample fluoroscopic image of a heart with a lasso catheter placed around a vein edge according to an exemplary embodiment of the present invention;

FIG. 3 is a sample fluoroscope image wherein the three-dimensional locations of edges of pulmonary veins are implied within a two-dimensional fluoroscope image with the use of four ellipses according to an exemplary embodiment of the present invention;

FIG. 4 is a sample fluoroscope image wherein the three-dimensional location of the lasso catheter electrodes around the edges of pulmonary veins are implied within a two-dimensional fluoroscope image with the use of points and text, according to an exemplary embodiment of the present invention and the chart demonstrates a possible usage for the text to identify an electrode with the signal it measures;

FIG. 5 is a sample fluoroscope image wherein the three-dimensional location of the lasso catheter electrodes around the edges of pulmonary veins are used to construct a virtual representation of structural data within a two-dimensional fluoroscope image according to an exemplary embodiment of the present invention;

FIG. 6 is a flow chart for visualization using a rotatable x-ray detector fluoroscope according to an exemplary embodiment of the present invention;

FIG. 7 is a flow chart illustrating a method for performing breathing compensation in a fused fluoroscope image using a monoplane system according to an exemplary embodiment of the present invention;

FIG. 8 is a flow chart illustrating a method for performing breathing compensation in a fused fluoroscope image using a bi-plane system according to an exemplary embodiment of the present invention; and

FIG. 9 shows an example of a computer system capable of implementing the method and apparatus according to embodiments of the present disclosure.

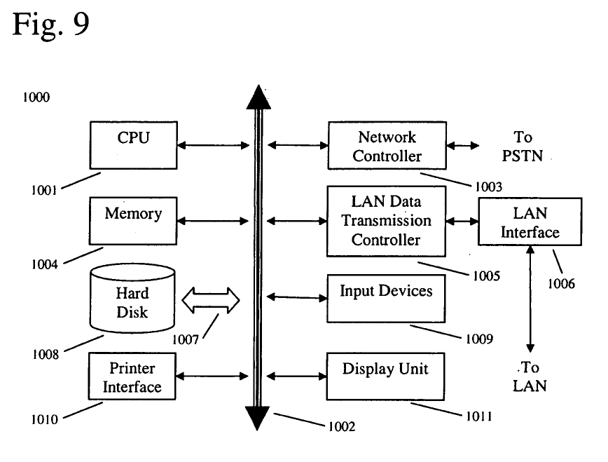

### DETAILED DESCRIPTION OF THE DRAWINGS

In describing exemplary embodiments of the present disclosure illustrated in the drawings, specific terminology is employed for sake of clarity. However, the present disclosure is not intended to be limited to the specific terminology so selected, and it is to be understood that each specific element includes all technical equivalents which operate in a similar manner.

Exemplary embodiments of the present invention may serve three purposes. First, exemplary embodiments of the present invention may seek to provide an approach for assisting a medical practitioner in performing invasive EP procedures and/or RF catheter ablation while using two-dimensional fluoroscope imagery. Here the two-dimensional fluoroscope imagery may be substantially unfused with structural volume data acquired using an MR or CT scan, although such volume data may be used to provide additional structural detail. Assistance may be in the form of one or more indicators or markers superimposed over the real-time fluoroscope moving image data that provide the medical practitioner with a visual clue that is suggestive of a sense of location and depth of desired structural elements so that the medical practitioner can more easily interact with anatomical structures such as, for example, the pulmonary veins, even when these structural elements would be difficult to see given the limitations of fluoroscopy in the imaging of soft tissue structures and the inability of fluoroscopy to provide a sense of depth.

Second, exemplary embodiments of the present invention may seek to provide an approach for maintaining precise registration of the fused image data in a manner that corrects for motion caused by breathing so that the three-dimensional structural detail can remain correctly registered to the real-time motion fluoroscope imagery during multiple phases of the respiratory cycle.

Third, exemplary embodiments of the present invention may seek to provide an approach for performing initial registration of fluoroscope imagery to a three-dimensional image volume such as, for example, an MR and/or CT scan so that fused imagery may be provided to the medical practitioner such that invasive EP procedures and/or RF catheter ablation may be performed with the assistance of real-time imagery that includes three-dimensional structural detail.

Visualization of Pulmonary Veins using Unfused Fluoroscope Imagery

While fused fluoroscope imagery may be used to provide a high level of structural detail in the imaging of the heart that may be particularly useful in performing invasive PE procedures and/or RF catheter ablation, fused fluoroscope imagery is not always practical or desirable. For this reason, exemplary embodiments of the present invention seek to overcome the problems discussed above related to the inability of fluoroscopy to accurately image soft tissue and display depth such that the medical practitioner may more easily interact with the desired structural elements such as, for example, the pulmonary veins, while using fluoroscope imagery that does not require the registration of volume image data.

In performing exemplary embodiments of the present invention, either a bi-plane x-ray detector or a monoplane x-ray detector may be used. The bi-plane x-ray detector is a device that is able to simultaneously (or nearly simultaneously) provide fluoroscope imagery from two distinct angles. Such devices are often used to provide the medical practitioner with multiple distinct views thereby increasing the likelihood of finding an optimum viewing angle during the performance of the invasive EP procedures and/or the RF catheter ablation.

Exemplary embodiments of the present invention may be used to reconstruct the three-dimensional location of pulmonary vein edges from two-dimensional fluoroscope imagery by relying on two distinct views that may be achieved either by moving a single x-ray detector or by using two x-ray detectors at different angles. This three-dimensional location information may be known for the entire cardiac cycle, and thus may contain location information that changes throughout the cardiac cycle. This positional data is often considered to be “4D” location information for this reason. However, for the purposes of simplicity, this location data is generally referred to herein as three-dimensional location information, and it is to be understood that this information may include data for the entire cardiac cycle (4D), where available.

The monoplane x-ray detector may have a c-arm configuration where the x-ray detector can be rotated about the subject so that a desired viewing angle may be selected. Alternatively, the monoplane x-ray detector may be stationary thereby providing only a single viewing angle.

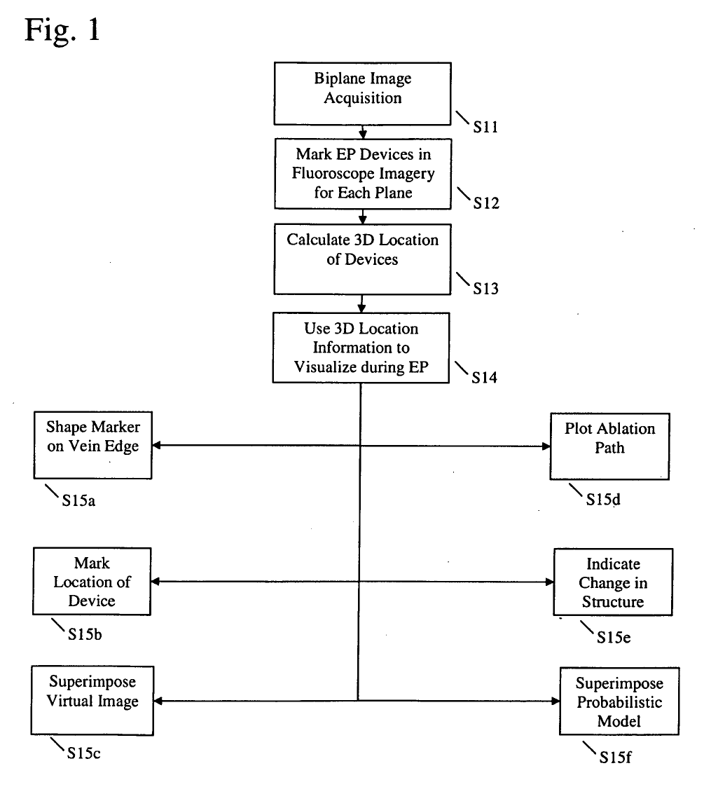

FIG. 1 is a flow chart illustrating a method for visualization using a bi-plane fluoroscope according to an exemplary embodiment of the present invention. First, bi-plane image acquisition may begin (Step S11). Bi-plane image acquisition may include the simultaneous or nearly simultaneous real-time moving fluoroscope imaging of a subject from two distinct angles.

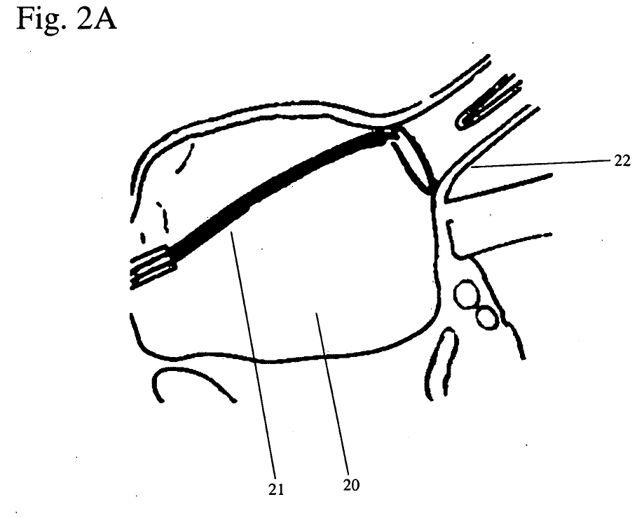

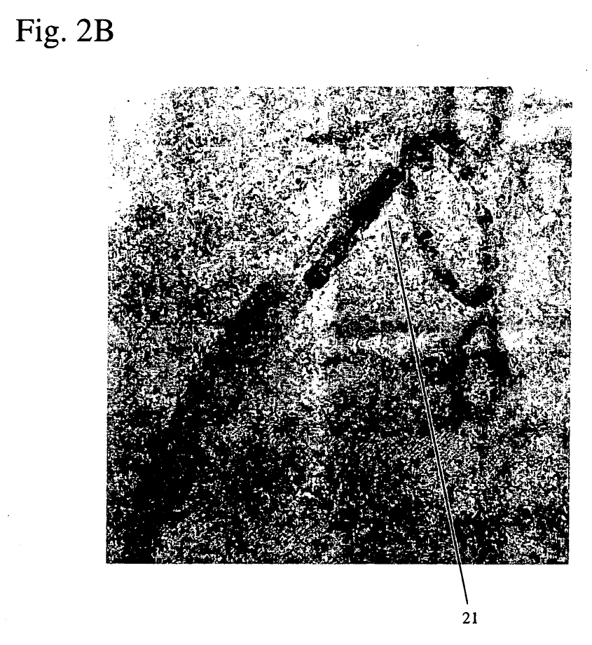

In performing invasive EP procedures, an EP instrument with a curving part such as a lasso catheter may be used. The lasso catheter may be used to electronically isolate various structural features. For example, when electronically isolating the pulmonary vein from the left atrium, the lasso catheter may be placed on one or more pulmonary veins connection with the left atrium. The placement of the lasso catheter may be used to designate a pulmonary vein edge. FIG. 2A is a diagram of a left atrium of a heart 20 with a lasso catheter 21 placed on a pulmonary vein edge 22. While the soft tissue comprising the heart at the pulmonary veins may be difficult to see within the fluoroscopic imagery, the lasso catheter may be easily visible. FIG. 2B is a sample fluoroscopic image of a heart with a lasso catheter placed around a vein edge. As can be seen from the image, the lasso catheter 21 may be clearly visible, while the surrounding soft tissue is more difficult to see.

When the lasso catheter is placed on the edge of a pulmonary vein, the bi-plane fluoroscope images the lasso catheter from two directions. Next, the loop of the lasso catheter may be marked on each of the bi-plane fluoroscope images, either manually or automatically (Step S12). Where the lasso catheter is marked manually, the medical practitioner may look at the fluoroscope images and indicate, using a suitable computer interface, the location of the lasso catheter. Where marking is automatic, image processing and vision techniques may be used to mark the lasso catheter.

Where there are multiple lasso catheters, for example, where multiple lasso catheters are placed around multiple pulmonary veins, marking may be performed for each lasso catheter. However, when there are multiple lasso catheters, the same lasso catheter may be similarly marked for each of the bi-plane images so that image data relating to the same lasso catheter from multiple angles are cross-referenced.

Next, the three-dimensional location of the loop part of the lasso catheter may be calculated for each lasso catheter (Step S13). This three-dimensional location may be calculated based on the angulations and x-ray system parameters from the bi-plane fluoroscope. Known computer vision techniques may be used to construct the three-dimensional location of the loops from the available bi-plane data.

Once the three-dimensional location of the pulmonary veins with lasso catheters on their edges have been calculated, this location information may be used in various ways to provide visual aid and guidance for EP procedures (Step S14). Six distinct indication techniques are described herein by way of example; however, other visualization techniques may also be performed. These techniques may use the available X-ray image geometry (i.e. projection matrices) to correctly perform visualization operations. These techniques may also utilize ECG signals and breathing indicators to better perform visualization. These techniques do not represent mutually exclusive approaches and thus these techniques may be practiced either by themselves or along with one or more other approaches.

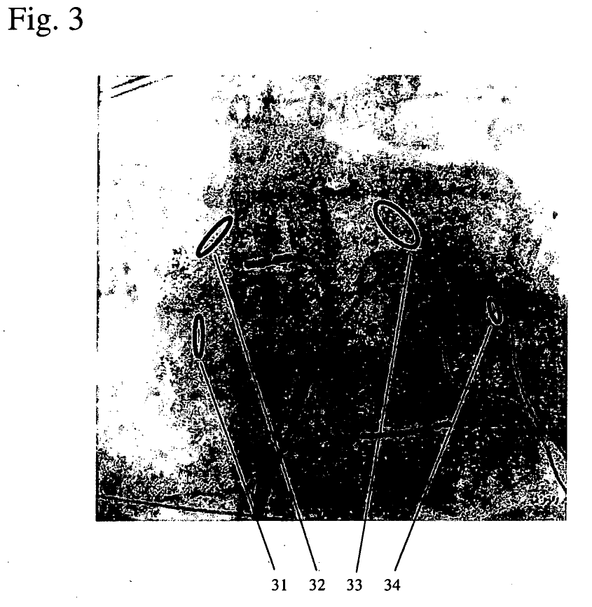

In the first such approach, one or more pulmonary vein edges overlaying X-ray images may be consistently outlined by using graphics such as (but not limited to) lines, curves, dots marking the edges, shapes (e.g. ellipse) representing the pulmonary vein, 2D/3D drawing and/or text indicating the edges. Thus, the three-dimensional location information pertaining to each pulmonary vein edge may be implied within one or both of the bi-plane two-dimensional fluoroscope images using a shape marker (Step S15a). FIG. 3 is a sample fluoroscope image wherein the three-dimensional location of edges of pulmonary veins are implied within a two-dimensional fluoroscope image with the use of four ellipses 31, 32, 33, and 34. An ellipse may serve as a particularly suitable example of an indication of the three-dimensional location of a pulmonary edge vein because the ellipse may be conceptualized as a two-dimensional projection of a circle that exists in three-dimensional space indicating the circumference of each pulmonary vein. However, other manners of indication, for example, those listed above, may also be well suited to the task of conveying a sense of depth to the medical practitioner relying on the fluoroscope image data in performing invasive PE procedures.

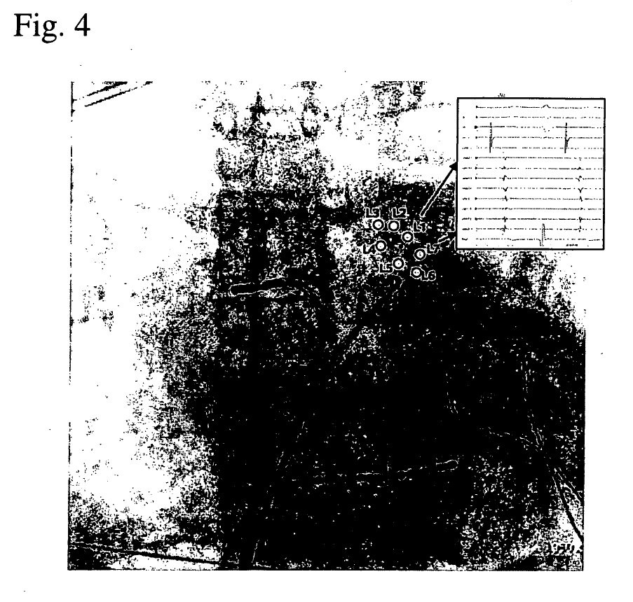

In the second approach for using the calculated three-dimensional location data of the conveying a sense of depth on the two-dimensional fluoroscope image, one or more reconstructed pulmonary vein edges may be used to consistently visualize a lasso catheter electrodes or electric mappings by using graphics such as (but not limited to) lines, curves, dots, shapes, drawing of whole/part of the lasso catheter (such as electrodes locations), and/or text indicating the three dimensional location of the lasso catheter placed on the pulmonary vein edge. Thus, rather than indicating the pulmonary vein edge its self, here the location of the lasso catheter around the vein edge may be expressed (Step S15b). FIG. 4 is a sample fluoroscope image wherein the three-dimensional location of the lasso catheter electrodes around the edges of pulmonary veins are implied within a two-dimensional fluoroscope image with the use of points and text, according to an exemplary embodiment of the present invention. This chart demonstrates a possible usage for the text to identify an electrode with the signal it measures. In this example, the text is used to indicate the different electrodes of the catheter: L1, L2, L3, L4, L5, L6, and L7. As shown, the matching electrode signals detected on the electrodes are also represented on the image. The location of the lasso catheter around the vein edge may continue to be shown even if the lasso catheters are subsequently moved from the pulmonary veins, as is the case in the image of FIG. 4.

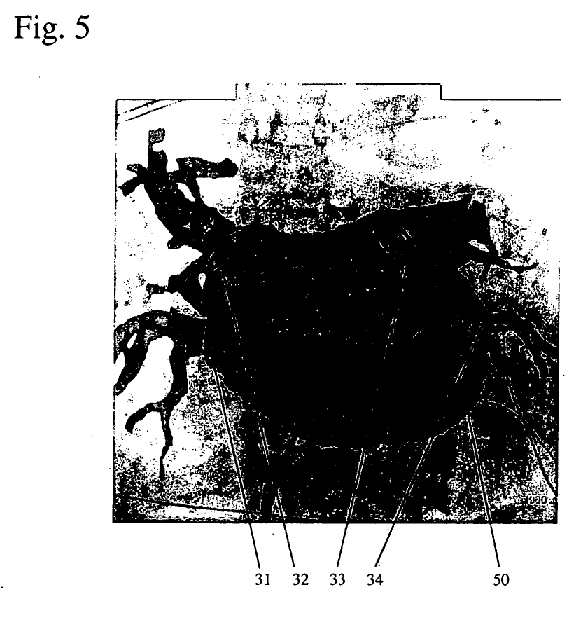

In the third approach, one or more reconstructed pulmonary vein edges may be superimposed over X-ray images or a video image, for example a sequence of images. The superimposed image/video may be, for example, a previous or live X-ray image/video, a photograph, a computer rendered image/video, a computer processed image/video, or/and graphics based on an image/video. The displayed image/video may be partially transparent, may contain an alpha channel and/or may be segments of an image/video. In this way, the three-dimensional location information for the lasso catheters may be used to superimpose an image of an anatomical structure, either actual or representative) that would aid a medical practitioner in gaining an understanding for one or more of the structural features that are not clearly visible from the fluoroscope imagery (Step S15c). For example, a three-dimensional segmentation/volume may be rendered and overlaid in a way it match the current estimated location in three-dimensions and displayed in a partially transparent way upon the current X-ray images. FIG. 5 is a sample fluoroscope image wherein the three-dimensional location of the lasso catheter electrodes around the edges of pulmonary veins are used to construct a virtual representation of structural data 50 within a two-dimensional fluoroscope image according to an exemplary embodiment of the present invention.

In the fourth approach, the three-dimensional location information for the pulmonary vein edges may be used to directly or indirectly plan an ablation plane and/or path along which to apply RF catheter ablation (Step S15d). The planned ablation plane/path may be superimposed over the fluoroscope imagery and may remain superimposed thereon from frame-to-frame of the fluoroscope imagery. Thus, the planned ablation plane/path may represent an illustration and/or graphic representation of where to place the ablation catheter that is superimposed over the fluoroscope imagery, for example, a trail of dots may be placed on the fluoroscopy images indicating where to ablate.

In the fifth approach, the three-dimensional lasso catheter location information may be used to register or align the fluoroscope imagery to a volume, segmentation or model. Registration may be performed manually, interactively, semi-automatically or fully automatically. Registration may be either rigid or non-rigid and may be based on the absolute or relative locations of the pulmonary vein edges, as determined by the three-dimensional lasso catheter location information. The registration maybe used by another application or device. For example, CT volume data may be non-rigidly registered to the fluoroscope imagery. The volume data may then be wrapped to match changes in the location of the pulmonary vein edges. Accordingly, changes to the anatomical structure may be indicated or animated within the fluoroscope imagery (Step S15e).

In the sixth approach, the three-dimensional lasso catheter location information may be used to match or create a probabilistic model of anatomical features, for example, a probabilistic model of the heart, and superimpose the model over the fluoroscope imagery (Step S15f). The probabilistic model of the heart may be constructed, for example, from the average radius and distance of the pulmonary vein edges, as determined from the three-dimensional lasso catheter location information.

As discussed above, in performing exemplary embodiments of the present invention, either a bi-plane x-ray detector or a monoplane x-ray detector may be used. When a monoplane x-ray detector is used, the x-ray detector may either be fully stationary, or movable and thus capable of capturing images from more than one plane. When an adjustable x-ray detector is used, the x-ray detector may be mounted to a c-arm unit so that the x-ray detector can be effectively rotated about the subject. Thus the c-arm mounted x-ray detector is capable of obtaining fluoroscope imagery from multiple planes; however, unlike the case for the bi-plane x-ray detector discussed above, the rotatable x-ray detector can only capture imagery from one plane at a time.

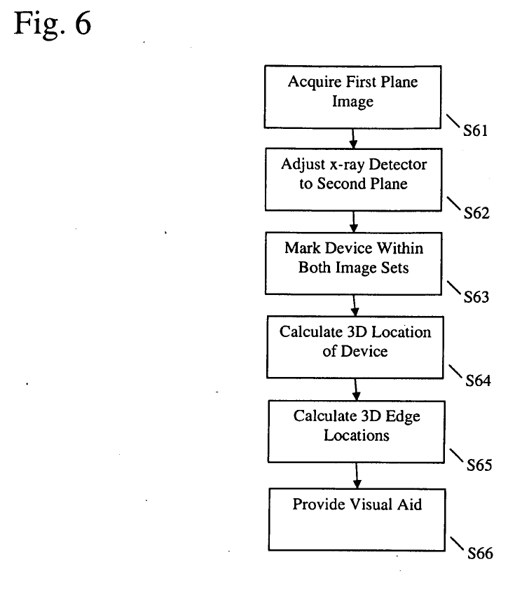

Thus the method for visualization described above with respect to FIG. 1 may be adapted for use with a rotatable x-ray detector. FIG. 6 is a flow chart for visualization using a rotatable x-ray detector fluoroscope according to an exemplary embodiment of the present invention. First, a first-plane image may be acquired (Step S61). The first-plane image may be a single frame x-ray image or may include multiple fluoroscopic image frames. The first-plane image may be captured from a plane that is not the desired plane in which to capture real-time imagery to be used during the invasive PE procedure. Next, the x-ray detector may be adjusted to capture imagery from a second plane that is not the same as the first plane (Step S62). To adjust the x-ray detector to the second plane, the x-ray detector may be rotated, for example, using the c-arm. The second plane may be the desired plane in which to capture real-time imagery to be used during the invasive PE procedure. The patient subject may remain motionless between the acquisition at the first plane (Step S61) and the acquisition at the second plane (Step S62). The loop of the lasso catheter may then be marked on the image data captured from both planes in a manner substantially similar that described above with respect to the bi-plane fluoroscope (Step S63). Then, the three-dimensional location of the loop part of the lasso catheter may be calculated from the marked image data in a manner substantially similar that described above with respect to the bi-plane fluoroscope (Step S64). Thereafter, the three-dimensional location of the pulmonary veins with lasso catheters on their edges may be calculated from the three-dimensional location information, also similar to the manner described above (Step S65), this location information may be used in various ways to provide visual aid and guidance for EP procedures (Step S66) using one or more of the approaches discussed above.

In many EP procedures, movement of the x-ray detector is either not possible or not practicable. Accordingly, in such a case, the three-dimensional location of the pulmonary veins may not be precisely discoverable. Even without this information, approaches one through four, discussed in detail above, may be performed by applying an assumption as to the three-dimensional location of the pulmonary veins. However, as this assumption is based on the precise location of the x-ray detector and patient subject, neither the detector nor the subject should be moved during the EP procedure.

The visual aids, for example, the ellipses drawn over the pulmonary vein edges, may be utilized with or without constructing the three-dimensional location of the vein edges. Accordingly, the vein edge may be indicated by a visual aid based on only the fluoroscope imagery from a single x-ray detector acquiring imagery from a single angle/plane. This may be performed by ensuring that the x-ray detector and patient subject remain stationary. In this case, the marker, for example, an ellipse, may be two-dimensional rather than three-dimensional.

In case of the visual aids the 3D location is not assumed. Just 2D visualizations are used (as if some drawn with a marker on the monitor).

### Resperatory Motion Compensation in Fused Fluoroscope Imagery

As discussed above, two-dimensional fluoroscopic images may lack detailed anatomical information due to the limitations of the X-ray detector in distinguishing among soft tissues. Recently, fused visualization of high-resolution three-dimensional atrial CT and/or MR volumes with fluoroscopic images has been used to provide a more realistic picture of a patient's heart anatomy, representing a major technological advance in diagnosing and treating complex arrhythmias

The high-resolution CT and/or MR volumes may be acquired preoperatively. These volumes may be acquired at a given cardiac and respiratory phase, and hence are fused (registered) correctly with the fluoroscopy (patient) only for that particular cardiac and respiratory state. Thus, a medical practitioner relying on the fused imagery in performing invasive PE procedures may encounter a situation in which the three-dimensional volume image data becomes periodically misaligned as the cardiac and respiratory cycle progresses. The medical practitioner may find this to be rather disconcerting. While cardiac motion could be compensated using ECG gating, breathing motion is less periodic than cardiac motion and is hence more difficult to compensate for.

Exemplary embodiments of the present invention relate to techniques for compensating for respiratory motion in fluoroscopic images that are fused with three-dimensional volume data. These techniques may utilize the fact that the devices that are routinely used during EP procedures such as AFIB ablation may be clearly discernable from within the fluoroscopy images, and thus the prominence of the inserted devices may be used for tracking and subsequent motion estimation. This disclosure discusses the use of lasso catheters and coronary sinus (CS) catheters as the EP procedure devices; however, exemplary embodiments of the present invention may also use other devices for this purpose, especially where they possess similar properties as the lasso and CS catheters. These properties include (1) they are put at a relatively fixed position and not frequently moved during the EP procedure, (2) movement of the devices is primarily attributable to the cardiac and respiratory motion, and (3) movement of the devices either closely represents or is in a synchronized fashion with the breathing motion.

The motion at left atrium and pulmonary vein due to breathing is largely translational and dominantly rigid-body up to ostia level. Accordingly, exemplary embodiments of the present invention may focus on compensating for translational and/or rigid-body motion.

As discussed above, fluoroscope imagery may be acquired with either a monoplane x-ray detector or a bi-plane detector. Furthermore, a wide variety of EP devices may be used. For descriptive purpose, the present disclosure may focus on lasso catheter for monoplane system and CS catheter for biplane system for three-dimensional breathing motion estimation and compensation. It should be understood, however, that any device may be used with any system.

Fluoroscope systems with monoplane detectors may be less expensive and hence more widely used than biplane systems. In order to be able to compensate for three-dimensional breathing motion using monoplane system, a patient-specific three-dimensional translational and/or rigid-body motion model may be constructed preoperatively. As in the case described above for monoplane pulmonary vein edge demarcation, the three-dimensional model may be constructed from two different views on monoplane system. These views may be non-synchronized but ECG-gated fluoroscopic sequences acquired from two distinct angles, for example, obtained by acquiring a first image from a first plane, repositioning the detector, and then acquiring a second image from a second plane.

The motion model need not be constructed on biplane system with two synchronized biplane views available at all times; the device may be further tracked directly in three-dimensions rather than in two-dimensions from the two-dimensional fluoroscope image data and the constructed motion model. This may potentially lead to more accurate and robust tracking.

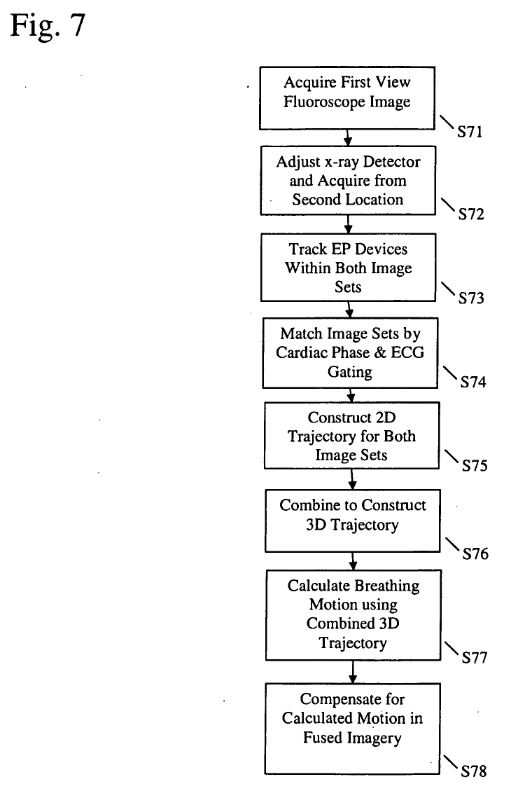

FIG. 7 is a flow chart illustrating a method for performing breathing compensation in a fused fluoroscope image using a monoplane system according to an exemplary embodiment of the present invention. First, a fluoroscopic sequence may be acquired from a first view while the patient subject is permitted to breathe freely (Step S71). Deep inhalation and exhalation may be recommended in order to cover the largest possible range of patient breathing. The fluoroscopic sequence may contain a sufficient number of different breathing states during the breathing cycle for a given cardiac phase. For example, the acquisition may last from 10 to 15 seconds and may cover 2 to 3 breathing cycles and 10 to 15 cardiac cycles.

Next, the x-ray detector may be repositioned to a second view and a second fluoroscopic sequence may be acquired (Step S72). The second view may be achieved by rotating a c-arm mounted x-ray detector. The second view may be at least 40 degrees apart from the view first. The second fluoroscopic sequence may be acquired with the same requirement and/or parameters as those used for the first sequence.

One or more lasso catheters may be tracked throughout both fluoroscopic sequences, for example, by performing robust ellipse fitting (Step S73). The cardiac phase may then be calculated using ECG signals and frames from both fluoroscopic sequences may be selected such that the frames represent approximately the same or similar cardiac phases as the cardiac phase within which the preoperative volume was acquired (Step S74).

For the ECG gated frames selected in step S74, the two-dimensional moving trajectory of the center of the loop of the lasso catheter during the whole breathing cycle may be constructed by interpolating the centers of the tracked loops (Step S75). Here, two distinct moving trajectories may be constructed, one for each plane sequence.

Next, a three-dimensional moving trajectory may be constructed for the center of the loop of the lasso catheter during the whole breathing cycle using the two two-dimensional moving trajectories constructed in Step S75 (Step S76). Here, an epipolar line constraint may be used in the construction of the three-dimensional trajectory. The three-dimensional moving trajectory may represent the three-dimensional translational motion model of the left atrium during the whole breathing cycle at a particular cardiac phase. For example, the cardiac phase of the left atrium may be the cardiac phase during which the CT and/or MR volume data was acquired.

The c-arm of the x-ray detector may then be adjusted to obtain a desired working position for performing the EP procedures, where the x-ray detector is not already in a suitable position. The three-dimensional translational motion to be compensated for may be calculated by tracking the lasso catheter on the fluoroscopy at that particular cardiac phase, back projecting the center of the loop of the lasso catheter onto the three-dimensional trajectory model constructed in Step S76, and finding the best match, for example using epipolar line constraint, (Step S77). This calculated translational motion may be the breathing motion.

After the translational motion to be compensated for is calculated in Step S77, the fused preoperative three-dimensional volume may then be moved according to the calculated translational motion (Step S78) and thus, breathing motion may be corrected for and the three-dimensional volume and the fluoroscope imagery may remain accurately registered throughout the respiratory cycle.

Various modifications may be made to the above-described procedure without departing from the scope of the invention. For example, rather than calculating the three-dimensional moving trajectory of the center of the loop of the lasso catheter in step S73, the three-dimensional moving trajectory may be calculated for the whole lasso catheter. To accomplish this, the position of the whole lasso catheter may be reconstructed by tracking the densely placed electrodes on the lasso catheter, reconstructing the moving trajectories of multiple electrodes and interpolating for the points between the electrodes. The three-dimensional rigid motion may then be compensated with the minimum number of three tracked electrodes.

Additionally, a breathing motion model may be learned for any cardiac phase from the two fluoroscopic sequences. When the three-dimensional volumetric data at multiple cardiac phases is available, breathing motion compensation can be applied for fused visualization with fluoroscopy taken at multiple cardiac phases by using the three-dimensional volume and the breathing motion model for the corresponding cardiac phase.

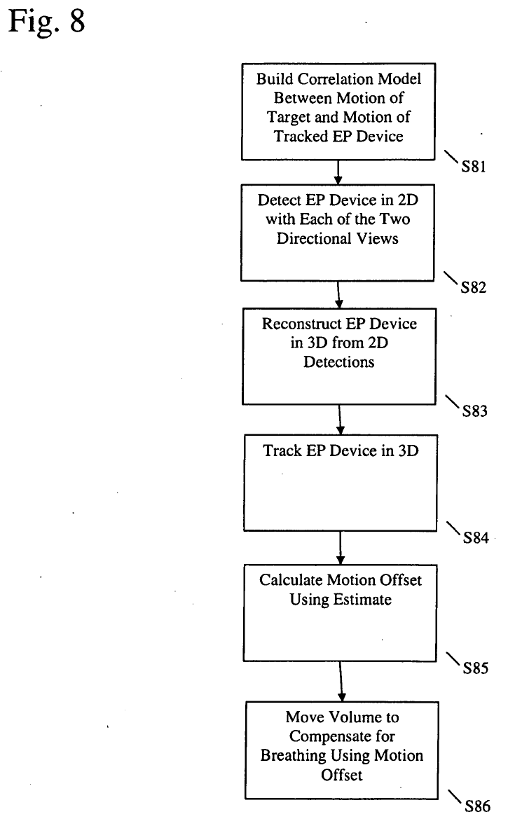

FIG. 8 is a flow chart illustrating a method for performing breathing compensation in a fused fluoroscope image using a bi-plane system according to an exemplary embodiment of the present invention. Here, the method may be discussed in terms of tracking a CS catheter, however, it is to be understood that any EP device may be used for tracking purposes. Details from the description above may be omitted for simplicity, but it is to be understood that aspects of the method described above may be combined with aspects of the method described below.

First, a correlation model may be built to relate estimated motion of the left atrium and motion of the CS catheter due to breathing (Step S81) in each two-dimensional view. The correlation model may be formed from statistics over a population of patients, or may be patient-specific. The motion of the left atrium and the CS catheter may be estimated using similar or distinct techniques. For example, magnetic tracking or image-based tracking of markers, for example, ablation catheters, may be directed to the particular target, for example, temporarily during motion data acquisition. The correlation model may include auto regression (AR) model, state-space model, neural network etc.

Next, the CS catheter may be detected within both x-ray detector views for the first frame (Step S82). CS catheter detection may be fully automatic or may involve manual interaction, for example, the user may use a mouse and cursor to double-click at each end of the CS catheter displayed on-screen.

The CS catheter may then be reconstructed in three-dimensions from the two-dimensional estimated motion of the CS catheter built in Step S81 (Step S83).

The CS catheter may then be tracked in three-dimensions so that the projection of the tracked catheter overlays with the moving CS catheters shown in the two biplane fluoroscopic sequences (Step S84).

Translational and/or rigid-body motion of the left atrium may then be calculated using the motion estimates and correlation model from Step S81 between the motion of left atrium and that of the CS catheter (Step S85). This calculated motion may be the estimated offset for the respiratory motion. The fused preoperative 3D volume may then be moved according to the estimated three-dimensional motion of the left atrium as calculated in Step S85 (Step S86).

Various modifications may be made to the above-described procedure without departing from the scope of the invention. For example, three-dimensional motion may be learned for various pulmonary veins and different parts of the left atrium by performing three-dimensional tracking of the devices that are temporarily located to the target position. The three-dimensional motion estimated from tracking can be a combination of cardiac and breathing motion, and may be further parameterized to provide an independent model for cardiac and breathing motion. An alternative is to isolate cardiac motion by ECG gating and build breathing motion model from the ECG gated tracking.

The correlation model may also be learned based on the relationship between the motions of different pulmonary veins and different parts of the left atrium, to provide quantitative analysis about the influence of breathing and cardiac motion on the anatomical change of the left atrium and pulmonary veins.

Exemplary embodiments of the present invention may thereby compensate for breathing motion in three-dimensions rather than simply trying to compensate for motion in two-dimensions, for both monoplane and biplane systems. By compensating for breathing motion in three-dimensions, adequate breathing motion compensation for the left atrium during EP applications may be performed.

By learning the breathing motion model in three-dimensions, exemplary embodiments of the present invention may be used to facilitate breathing motion compensation for any working angle, and thus, the working angle may even be adjusted during the course of the EP procedure.

Because motion compensation is performed based on devices that are routinely used during EP procedures, such as CS catheters and lasso catheters, additional markers need not be implanted into patients. Accordingly, image-based device tracking is performed on fluoroscopic images that are routinely used during EP procedures for monitoring and navigation, and additional detection hardware is not required. Thus, exemplary embodiments of the present invention may be workflow-friendly and cost-effective. Moreover, contrast agent administration is not required. Exemplary embodiments of the present invention may also be fully automatic and may be performed without user interaction.

### Initial Registration in Resperatory Motion Compensation in Fused Fluoroscope Imagery

Before three-dimensional tracking and estimation of the EP devises may be performed, an initial registration may be performed to relate the fluoroscope imagery to the CT and/or MR volume data. This initial registration may be performed in any number of ways, for example, registration may be performed by referencing the edges of the pulmonary veins in the volume data to the location of the edges pulmonary veins in the fluoroscope data. However, because the edges of the pulmonary veins are not easily detectible from within the fluoroscope data, and because three-dimensional location information of the pulmonary veins can not ordinarily be determined from the fluoroscope imagery, exemplary embodiments of the present invention may perform initial registration of the fused fluoroscope imagery by first identifying the edges of the pulmonary veins in three-dimensions form the fluoroscope imagery using one or more of the techniques discussed above with respect to FIGS. 1-7.

FIG. 10 shows an example of a computer system which may implement a method and system of the present disclosure. The system and method of the present disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

The computer system referred to generally as system 1000 may include, for example, a central processing unit (CPU) 1001, random access memory (RAM) 1004, a printer interface 1010, a display unit 1011, a local area network (LAN) data transmission controller 1005, a LAN interface 1006, a network controller 1003, an internal bus 1002, and one or more input devices 1009, for example, a keyboard, mouse etc. As shown, the system 1000 may be connected to a data storage device, for example, a hard disk, 1008 via a link 1007.

Exemplary embodiments described herein are illustrative, and many variations can be introduced without departing from the spirit of the disclosure or from the scope of the appended claims. For example, elements and/or features of different exemplary embodiments may be combined with each other and/or substituted for each other within the scope of this disclosure and appended claims.

What is claimed is:

1. A method for real-time cardiac visualization, comprising:
acquiring fluoroscope imagery from two planes;
marking the location of at least one electrophysiology (EP) device within the fluoroscope imagery from each of the two planes;
combining the location information for the at least one EP device within each of the acquired fluoroscope images from the two planes to determine a 3D location for the at least one EP device; and
displaying the fluoroscope imagery from at least one of the two planes with a graphical visual aid superimposed thereon, the visual aid being based on the 3D location of the EP device.

2. The method of claim 1, wherein acquiring the fluoroscope imagery from two planes, comprises:
acquiring fluoroscope imagery from a first plane using an x-ray detector;
repositioning the x-ray detector to a second plane; and
acquiring fluoroscope imagery from the second plane using the repositioned x-ray detector.

3. The method of claim 1, wherein acquiring the fluoroscope imagery from two planes, comprises:
acquiring fluoroscope imagery from a first plane using a first x-ray detector; and
acquiring fluoroscope imagery from a second plane using a second x-ray detector, wherein the first x-ray detector and the second x-ray detector are part of a single biplane fluoroscope.

4. The method of claim 1, wherein the location of the at least one EP device is marked manually by a user who is presented with an on-screen representation of each fluoroscope image and selects the location of the EP device on each fluoroscope image or is marked semi-automatically with the use of an interactive tool.

5. The method of claim 1, wherein the location of the at least one EP device is marked automatically on each fluoroscope image using computer vision techniques.

6. The method of claim 1, wherein the at least one EP device includes a lasso catheter or a CS catheter.

7. The method of claim 1, wherein displaying the fluoroscope imagery from at least one of the two planes with a visual aid superimposed thereon includes displaying a shape marker indicating the 3D location of a pulmonary vein edge.

8. The method of claim 7, wherein the shape marker is an ellipse.

9. The method of claim 1, wherein displaying the fluoroscope imagery from at least one of the two planes with a visual aid superimposed thereon includes displaying a shape marker indicating the 3D location of the at least one EP device.

9. The method of claim 1, wherein displaying the fluoroscope imagery from at least one of the two planes with a visual aid superimposed thereon includes displaying a suggested ablation path.

10. The method of claim 1, wherein displaying the fluoroscope imagery from at least one of the two planes with a visual aid superimposed thereon includes displaying a rendered 3D segmentation of a left atrium.

11. A method for compensating for breathing motion in a real-time cardiac visualization, comprising:
acquiring fluoroscope imagery from two planes;
tracking at least one electrophysiology (EP) device within the acquired fluoroscope imagery from each of the two planes;
constructing a 2D trajectory for the at least one EP device within the acquired fluoroscope imagery from each of the two planes based on the tracking;
constructing a 3D trajectory for the at least one EP device by combining the 2D trajectories of the at least one EP device for each of the two planes;
determining a breathing motion based on the constructed 3D trajectory; and
compensating for the determined breathing motion within the acquired fluoroscope imagery.

12. The method of claim 11, wherein the acquired fluoroscope imagery is registered to 3D volume data acquired from a CT or MR and the fluoroscope imagery is fused to the registered 3D volume data such that the fused image data provides a real-time moving image with structural detail, and wherein the fused image data is compensated for by the determined breathing motion.

13. The method of claim 12, wherein fusing the fluoroscope imagery to the registered 3D volume data includes matching the fluoroscope imagery to the cardiac phase of the 3D volume data and performing ECG.

14. The method of claim 12, wherein performing initial registration of the fluoroscope imagery to the 3D volume data comprises:
marking the location of at least one EP device within the fluoroscope imagery from each of the two planes;
combining the location information for the at least one EP device within each of the acquired fluoroscope images from the two planes to determine a 3D location for the at least one EP device;
identifying a 3D location of an anatomical structure within the fluoroscope imagery based on the determined 3D location for the at least one EP device; and
registering the fluoroscope imagery to the 3D volume using the identified 3D location of the anatomical stricture.

15. The method of claim 11, wherein acquiring the fluoroscope imagery from two planes, comprises:
acquiring fluoroscope imagery from a first plane using an x-ray detector;
repositioning the x-ray detector to a second plane; and
acquiring fluoroscope imagery from the second plane using the repositioned x-ray detector.

16. The method of claim 11, wherein acquiring the fluoroscope imagery from two planes, comprises:
acquiring fluoroscope imagery from a first plane using a first x-ray detector; and
acquiring fluoroscope imagery from a second plane using a second x-ray detector, wherein the first x-ray detector and the second x-ray detector are part of a single biplane fluoroscope.

17. The method of claim 11, wherein the at least one EP device includes a lasso catheter or a CS catheter.

18. A computer system comprising:
a processor; and
a program storage device readable by the computer system, embodying a program of instructions executable by the processor to perform method steps for real-time cardiac visualization, the method comprising:
acquiring fluoroscope imagery from two planes;
marking the location of at least one lasso catheter within the fluoroscope imagery from each of the two planes;
combining the location information for the at least one lasso catheter within each of the acquired fluoroscope images from the two planes to determine a 3D location for the at least one lasso catheter;
determining the 3D location of one or more pulmonary vein edges based on the determined 3D location of the at least one lasso catheter, and
displaying the fluoroscope imagery from at least one of the two planes with an indication of the 3D location of the one or more pulmonary vein edges superimposed thereon.

19. The computer system of claim 18, wherein acquiring the fluoroscope imagery from two planes, comprises:
acquiring fluoroscope imagery from a first plane using an x-ray detector;
repositioning the x-ray detector to a second plane; and
acquiring fluoroscope imagery from the second plane using the repositioned x-ray detector.

20. The computer system of claim 18, wherein acquiring the fluoroscope imagery from two planes, comprises:
acquiring fluoroscope imagery from a first plane using a first x-ray detector; and
acquiring fluoroscope imagery from a second plane using a second x-ray detector, wherein the first x-ray detector and the second x-ray detector are part of a single biplane fluoroscope.

21. A method for real-time cardiac visualization, comprising:
acquiring fluoroscope imagery from a single plane with a stationary x-ray detector;
marking the location of at least one electrophysiology (EP) device within the fluoroscope imagery;
determining a location for the at least one EP device based on the fluoroscope imagery and a location of the stationary x-ray detector; and
displaying the fluoroscope imagery with a graphical visual aid superimposed thereon, the visual aid being based on the location of the EP device.

22. The method of claim 21, wherein the location of the at least one EP device is marked manually by a user who is presented with an on-screen representation of the fluoroscope image and selects the location of the EP device on the fluoroscope image or is marked semi-automatically with the use of an interactive tool.

23. The method of claim 21, wherein the location of the at least one EP device is marked automatically on the fluoroscope image using computer vision techniques.

24. The method of claim 21, wherein the at least one EP device includes a lasso catheter or a CS catheter.

25. The method of claim 21, wherein displaying the fluoroscope imagery with a visual aid superimposed thereon includes displaying a shape marker indicating the location of a pulmonary vein edge.

26. The method of claim 25, wherein the shape marker is an ellipse.
